(19) United States
(12) Patent Application Publication
(10) Pub. No.: US 2024/0233577 A9
(43) Pub. Date: Jul. 11, 2024

(54) METHOD AND SYSTEM FOR ASSESSING AN INJECTION OF A PHARMACEUTICAL PRODUCT

(71) Applicant: CAE HEALTHCARE CANADA INC., Saint-Laurent (CA)

(72) Inventors: Laurent DESMET, Saint-Laurent (CA); Caroline PERNELLE, Saint-Laurent (CA); Mona HALLAQ, Saint-Laurent (CA); Charlotte FOREST, Saint-Laurent (CA); Fan E, Saint-Laurent (CA)

(73) Assignee: CAE HEALTHCARE CANADA INC., Saint-Laurent, QC (CA)

(21) Appl. No.: 18/547,353

(22) PCT Filed: Feb. 22, 2022
(86) PCT No.: PCT/IB2022/051557

§ 371 Date: Aug. 22, 2023

Related U.S. Application Data
(60) Provisional application No. 63/152,132, filed on Feb. 22, 2021.

(51) Int. Cl. G09B 23/28; G06T 7/50; G06T 7/60; G06T 7/70

(52) U.S. Cl. CPC G09B 23/285; G06T 7/50; G06T 7/60; G06T 7/70; A61M 5/427

(57) ABSTRACT

In There is provided a computer-implemented method for assessing an injection performed on a subject using a syringe provided with a needle, the method comprising: processing a sequence of images taken of the injection for: determining an insertion angle of the needle relative to the subject; and determining a depth of insertion of the needle within the subject; determining one of a speed of injection and a duration of injection; and outputting an indication of the insertion angle, the depth of insertion and the one of the speed of injection and the duration of injection.

### TECHNICAL FIELD

The present invention relates to the field of methods and systems for assessing an injection of a pharmaceutical product, and more particularly to methods and systems for assessing an injection of a pharmaceutical product performed using a syringe.

### BACKGROUND

Medical syringe training is usually performed under the direct supervision of a professor or an experienced healthcare practitioner, a training approach that is expensive and human resource consuming.

Furthermore, since the evaluation is performed by a human being, errors may happen in the evaluation. For example, it may be difficult for a human being to evaluate whether the insertion angle of a needle within a subject is adequate.

Therefore, there is a need for an improved method and system for assessing the insertion of a needle and/or the injection of a pharmaceutical product using the needle and a syringe.

### SUMMARY

According to a first broad aspect, there is provided a computer-implemented method for assessing an injection performed on a subject using a syringe provided with a needle, the method comprising: processing a sequence of images taken of the injection for: determining an insertion angle of the needle relative to the subject; and determining a depth of insertion of the needle within the subject; determining one of a speed of injection and a duration of injection; and outputting an indication of the insertion angle, the depth of insertion and the one of the speed of injection and the duration of injection.

In one embodiment, the injection angle comprises an angle between a longitudinal axis of the syringe and a tangent line to a surface of the subject at a contact point between the needle and the surface of the subject.

In one embodiment, the step of determining an insertion angle comprises: identifying a given one of the images in which a distal end of the needle comes into contact with the subject; and measuring the insertion angle within the given one of the images.

In one embodiment, the step of said identifying the given one of the images comprises processing the sequence of images for: tracking the needle and the subject within the sequence of images; and identifying the given one of the images as being a first image in the sequence of images in which first coordinates of the distal end of the needle corresponds to second coordinates of a point of the surface of the subject. In another embodiment, the step of identifying the given one of the images comprises processing the sequence of images for: calculating a distance between a reference point located on one of the needle and the syringe and the surface of the subject; and identifying the given one of the images as being a first image in the sequence of images in which the calculated distance is one of equal to and less than a target distance.

In one embodiment, the step of determining an insertion angle comprises: identifying a plurality of the images in which a distal end of the needle comes into contact with the subject; measuring the respective insertion angle within each one of the plurality of the images; and calculating one of a median insertion angle and an average insertion angle based on the respective insertion angles, thereby obtaining the insertion angle.

In one embodiment, the step of determining a depth of insertion comprises processing the sequence of images for determining whether the needle has been entirely inserted into the subject.

In one embodiment, the step of determining a depth of insertion comprises processing the sequence of images for determining a given image in the sequence in which the needle has stopped moving along a longitudinal axis of the syringe.

In one embodiment, the step of determining a depth of insertion further comprises determining within the given image whether the needle is visible. In another embodiment, the step of determining a depth of insertion further comprises measuring, within the given image, a length of a visible portion of the needle. In a further embodiment, the step of determining a depth of insertion further comprises determining, within the given image, a position of a reference point on the syringe relative to a surface of the subject and calculating the depth of insertion based on the position of the reference point.

In one embodiment, the step of determining one of the speed of injection and the duration of injection comprises determining the duration of the injection.

In one embodiment, the duration of the injection corresponds to a time elapsed between a first image in which the needle comes into contact with the subject and a subsequent image in which the needle is no longer in contact with the subject. In another embodiment, the duration of the injection corresponds to a time elapsed between a first image in which the insertion depth has reached a desired depth and a subsequent image in which the needle is no longer in contact with the subject.

In one embodiment, the step of determining one of the speed of injection and the duration of injection comprises tracking a position of a plunger relative to a barrel of the syringe.

In one embodiment, the step of tracking is performed within the sequence of images.

In one embodiment, the method further comprises: determining whether the insertion angle is adequate, whether the depth of insertion is adequate and whether the one of the speed of injection and the duration of injection is adequate, thereby obtaining assessment results; and outputting the assessment results.

In one embodiment, the insertion angle is determined as being adequate if the insertion angle is comprised between a predefined minimal angle and a predefined maximal angle.

In one embodiment, the depth of insertion is determined as being adequate by determining that the syringe comes into contact with the subject. In another embodiment, the depth of insertion is determined as being adequate if the depth of insertion is comprised between a predefined minimal insertion and a predefined maximal insertion.

In one embodiment, the one of the speed of injection and the duration of injection is determined as being adequate if the one of the speed of injection and the duration of injection is comprised between a first threshold and a second threshold.

According to another broad aspect, there is provided a non-volatile memory having stored thereon statements and instructions that upon execution by a processor perform the steps of the above computer-implemented method.

According to a further broad aspect, there is provided a system for assessing an injection of a pharmaceutical product, the system comprising at least one processor and a memory, the memory having stored thereon statements and instructions that upon execution by the at least one processor perform the steps of: processing a sequence of images taken of the injection for: determining an insertion angle of the needle relative to the subject; and determining a depth of insertion of the needle within the subject; determining one of a speed of injection and a duration of injection; and outputting an indication of the insertion angle, the depth of insertion and the one of the speed of injection and the duration of injection.

In one embodiment, the injection angle comprises an angle between a longitudinal axis of the syringe and a tangent line to a surface of the subject at a contact point between the needle and the surface of the subject.

In one embodiment, the step of determining an insertion angle comprises: identifying a given one of the images in which a distal end of the needle comes into contact with the subject; and measuring the insertion angle within the given one of the images.

In one embodiment, the step of said identifying the given one of the images comprises processing the sequence of images for: tracking the needle and the subject within the sequence of images; and identifying the given one of the images as being a first image in the sequence of images in which first coordinates of the distal end of the needle corresponds to second coordinates of a point of the surface of the subject. In another embodiment, the step of identifying the given one of the images comprises processing the sequence of images for: calculating a distance between a reference point located on one of the needle and the syringe and the surface of the subject; and identifying the given one of the images as being a first image in the sequence of images in which the calculated distance is one of equal to and less than a target distance.

In one embodiment, the step of determining an insertion angle comprises: identifying a plurality of the images in which a distal end of the needle comes into contact with the subject; measuring the respective insertion angle within each one of the plurality of the images; and calculating one of a median insertion angle and an average insertion angle based on the respective insertion angles, thereby obtaining the insertion angle.

In one embodiment, the step of determining a depth of insertion comprises processing the sequence of images for determining whether the needle has been entirely inserted into the subject.

In one embodiment, the step of determining a depth of insertion comprises processing the sequence of images for determining a given image in the sequence in which the needle has stopped moving along a longitudinal axis of the syringe.

In one embodiment, the step of determining a depth of insertion further comprises determining within the given image whether the needle is visible. In another embodiment, the step of determining a depth of insertion further comprises measuring, within the given image, a length of a visible portion of the needle. In a further embodiment, the step of determining a depth of insertion further comprises determining, within the given image, a position of a reference point on the syringe relative to a surface of the subject and calculating the depth of insertion based on the position of the reference point.

In one embodiment, the step of determining one of the speed of injection and the duration of injection comprises determining the duration of the injection.

In one embodiment, the duration of the injection corresponds to a time elapsed between a first image in which the needle comes into contact with the subject and a subsequent image in which the needle is no longer in contact with the subject. In another embodiment, the duration of the injection corresponds to a time elapsed between a first image in which the insertion depth has reached a desired depth and a subsequent image in which the needle is no longer in contact with the subject.

In one embodiment, the step of determining one of the speed of injection and the duration of injection comprises tracking a position of a plunger relative to a barrel of the syringe.

In one embodiment, the step of tracking is performed within the sequence of images.

In one embodiment, the at least one processor is further configured for determining whether the insertion angle is adequate, whether the depth of insertion is adequate and whether the one of the speed of injection and the duration of injection is adequate, thereby obtaining assessment results; and outputting the assessment results.

In one embodiment, the insertion angle is determined as being adequate if the insertion angle is comprised between a predefined minimal angle and a predefined maximal angle.

In one embodiment, the depth of insertion is determined as being adequate by determining that the syringe comes into contact with the subject. In another embodiment, the depth of insertion is determined as being adequate if the depth of insertion is comprised between a predefined minimal insertion and a predefined maximal insertion.

In one embodiment, the one of the speed of injection and the duration of injection is determined as being adequate if the one of the speed of injection and the duration of injection is comprised between a first threshold and a second threshold.

According to still another broad aspect, there is provided a kit for assessing an injection performed using a syringe provided with a needle, the system comprising: a subject comprising an anatomical model; a support comprising an opening for receiving therein a camera configured for capturing a sequence of images of the injection performed on the anatomical model; and a syringe provided with a needle.

In one embodiment, the anatomical model is shaped so as to mimic a shape of a portion of a body of a human being.

In one embodiment, the support is adapted to provide the camera, when received in the support, with a predefined orientation relative to a receiving surface on which the support is to be deposited.

In one embodiment, the kit further comprises a mat for receiving the anatomical model and the support thereon, the mat comprising marks thereon for indicating at least one of a position and an orientation for the anatomical model and the support.

According to still a further embodiment, there is provided a method for assessing an injection performed on a subject using a syringe provided with a needle, the method comprising: performing the injection on the subject; concurrently taking a sequence of images of the performed injection; providing the sequence of images for processing to determine an insertion angle of the needle relative to the subject and a depth of insertion of the needle within the subject; and outputting an indication of the insertion angle and the depth of insertion.

### BRIEF DESCRIPTION OF THE DRAWINGS

Further features and advantages of the present invention will become apparent from the following detailed description, taken in combination with the appended drawings, in which:

FIG. 1 is a flow chart illustrating a method for assessing an injection performed using a syringe provided with a needle, in accordance with an embodiment;

FIG. 2A is an exemplary picture showing a needle approaching an anatomical model;

FIG. 2B is an exemplary picture showing the needle of FIG. 2A coming into contact with the anatomical model;

FIG. 2C is an exemplary picture showing the needle of FIG. 2A being entirely inserted into the anatomical model;

FIG. 3 illustrates a syringe provided with a needle, in accordance with the prior art;

FIG. 4 is a block diagram illustrating a system for assessing an injection performed using a syringe provided with a needle, in accordance with an embodiment;

FIG. 5 is a picture showing a system comprising a smartphone received in a support, an anatomical model and the syringe provided with a needle, in accordance with an embodiment;

FIG. 6A is a perspective view of the anatomical model of FIG. 5;

FIG. 6B is a side view of the anatomical model of FIG. 5;

FIG. 7 is a flowchart illustrating a method for assessing an injection performed using the syringe of FIG. 5, in accordance with an embodiment;

FIG. 8A is an exemplary graphical interface requesting a user to identify whether he is right-handed or left-handed;

FIG. 8B is an exemplary graphical interface illustrating instructions for assembling the system of FIG. 5 for a right-handed user;

FIG. 8C is an exemplary graphical interface illustrating instructions for assembling the system of FIG. 5 for a left-handed user;

FIG. 8D is an exemplary graphical interface for instructing a user to start recording a video;

FIG. 8E is an exemplary graphical interface showing negative assessment results;

FIG. 8F is an exemplary graphical interface showing positive assessment results;

FIG. 9 is an exemplary picture showing the anatomical model of FIG. 5 identified by a first bounding box;

FIG. 10 is an exemplary picture showing the anatomical model and the first bounding box of FIG. 9 and further showing a second bounding box defining a search area;

FIG. 11 is an exemplary picture showing the anatomical model and first bounding box of FIG. 9, the second bounding box of FIG. 10, as well as a syringe provided with a needle and identified by a third bounding box, the distal end of the needle coming into contact with the anatomical model; and

FIG. 12 is an exemplary picture showing the needle of FIG. 11 entirely inserted into the anatomical model.

It will be noted that throughout the appended drawings, like features are identified by like reference numerals.

### DETAILED DESCRIPTION

In the following there is provided a computer-implemented method for assessing an injection of a product in order to train a user to perform injections using a syringe provided with a needle. The method allows for automatically evaluating an injection performed by a user without the surveillance of a professor or an experienced healthcare practitioner. There is also provided a kit to be used in connection with the assessment method. The kit comprises an anatomical model on which the injection is to be performed and a support for receiving a camera to be used for capturing images of the injection in the anatomical model. The kit may further comprise a mat for receiving the anatomical model and the support thereon while ensuring a predefined relative position between the anatomical model and the support.

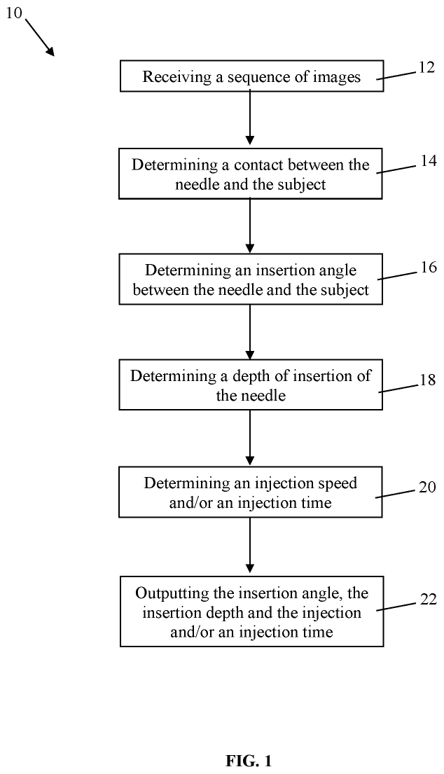

FIG. 1 illustrates one embodiment of a computer-implemented method 10 for assessing an injection of a pharmaceutical product to a subject performed by a user while using a syringe provided with a needle. While injecting a pharmaceutical product, the insertion angle of the needle, the insertion depth of the needle and the injection speed may be of importance. Therefore, assessing these parameters while a user such as a medical student performs an injection on a subject may be useful to evaluate and/or train the user. For example, a user may be instructed to perform an injection of a given volume of pharmaceutical product on a subject using a syringe provided with a needle. The user is instructed to perform the injection with a desired insertion angle, a desired depth of insertion of the needle into the subject and a desired injection speed or within a desired period of time. Images comprising at least the needle and at least part of the subject are captured while the user performs the injection on the subject and the images are analyzed to determine the insertion angle of the needle, the insertion depth and the speed or duration of injection. These determined injection parameters may then be analyzed to assess the performance of the user in performing the injection.

It should be understood that the method 10 may be used to assess the injection of any pharmaceutical product that can be injected into a subject using a needle mounted on a syringe. For example, the pharmaceutical product may be a biological product, a chemical product, a medicinal product, or the like. For example, the pharmaceutical product can be a vaccine, insulin, etc. In an embodiment in which the method is used in a context of training and the subject is inanimate, the pharmaceutical product may be any adequate fluidic product such as air, water, or the like.

At step 12, a sequence of images illustrating the insertion of a needle into a subject by a user and the injection of a pharmaceutical product into the subject is received. The images sequentially illustrate a needle secured to a syringe moving towards the surface of the subject, the distal end of the needle coming in contact with the surface of the subject, the needle being inserted into the subject, the actuation and displacement of the plunger of the syringe to deliver the pharmaceutical product. In one embodiment, the images further illustrate the extraction of the needle from the subject until the needle is no longer in contact with the subject. It should be understood that the received images are timely ordered so that the position of a given image within the sequence of images corresponds to a respective point in time during the administration of the pharmaceutical product since the number of images per second is fixed and known. In one embodiment each image of the sequence is time-stamped so that a temporal order is provided to the sequence of images. In the following, since the images are timely ordered, it should be understood that identifying or referring to a particular point in time is equivalent to identifying or referring to the corresponding image. For example, identifying the first point in time at which a needle comes in contact with the subject is equivalent to identifying the first image in the sequence in which the needle comes in contact with the subject, and vice-versa.

In one embodiment, the sequence of images are all received concurrently. For example, step 12 may consist in receiving a video file containing the sequence of images. In another embodiment, the images are iteratively received as they are being captured by a camera.

In one embodiment, the sequence of images are part of a video captured by at least one camera. As described below in greater detail, a single camera may be used to capture the insertion of the needle, the injection of the pharmaceutical product, and optionally the extraction of the needle. In another embodiment, at least two cameras may be used. In this case, step 12 comprises receiving a sequence of images from each camera and the received sequences of images all represent the same needle insertion and the same product injection but from different points of view or fields of view. For example, the cameras may be at different locations within a same plane or at different locations within different planes.

In one embodiment, the subject is an inanimate subject such as an object. For example, an inanimate object may be an anatomical model such as an object mimicking a part of a body such as a shoulder of a human being. In another embodiment, an inanimate object may be a fruit such as an orange. It should be understood that any adequate object in which a needle may be inserted may be used. For example, an inanimate object may be made of foam.

In another embodiment, the subject may be a living subject. For example, the subject may a human being, an animal such as a mammal, etc.

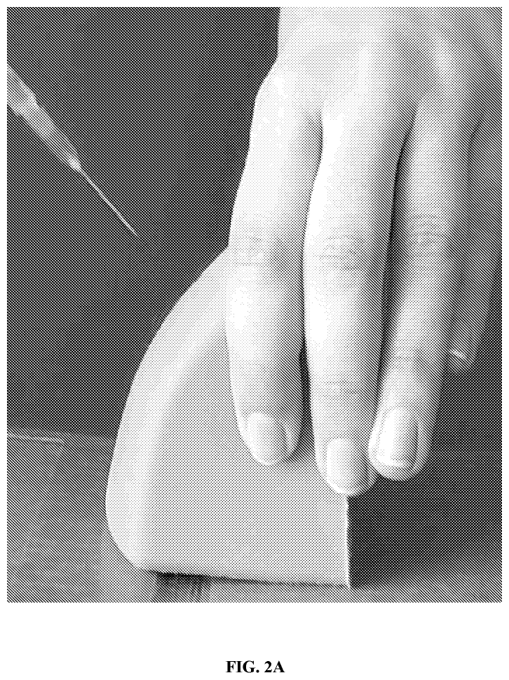

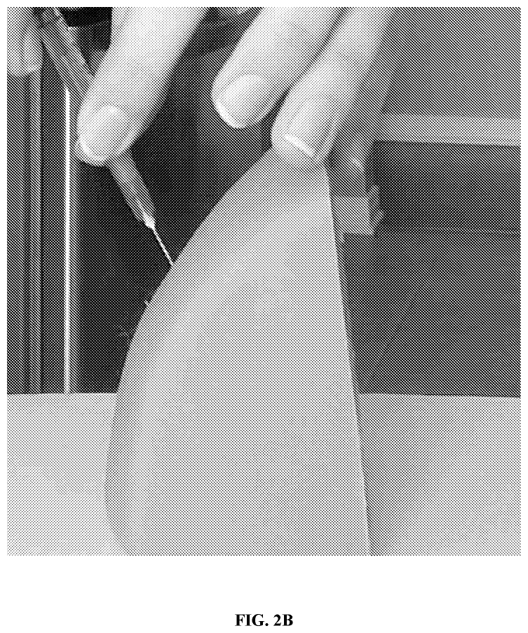

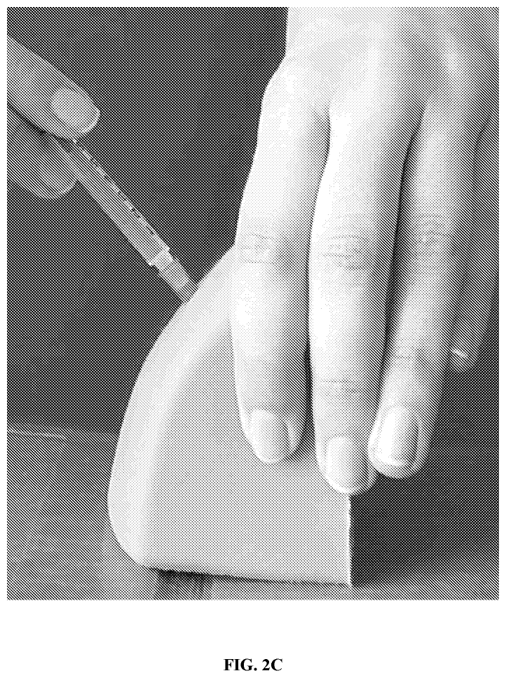

FIGS. 2A, 2B and 2C illustrates three exemplary images of a sequence of images that may be received at step 12. FIGS. 2A, 2B and 2C illustrates the insertion of a needle secured to a syringe into an inanimate subject at three different points in time. In FIG. 2A, a user holds a syringe having a needle secured thereto and the distal end of the needle is spaced apart from the inanimate subject. In FIG. 2B, the distal end of the syringe comes into contact with the surface of the inanimate subject. FIG. 2C illustrates the syringe when the whole length of the needle is inserted into the inanimate subject.

Referring back to FIG. 1, the second step 14 of method 10 comprises determining the point of contact between the distal end of the needle and the surface of the subject. The received images are iteratively analyzed starting from the first image of the sequence of images to determine whether the distal end of the needle is in physical contact with the subject, e.g., whether the distal end of the needle superimposes with a point of the surface of the subject. The first image in which the distal end of the needle is in physical contact with the subject marks the beginning of the insertion of the needle into the subject. For example, the first image in which the distal end of the needle superimposes with a point of the surface of the subject may correspond to the point in time at which the needle comes into contact with the subject, i.e., the beginning of the insertion of the needle into the subject.

It should be understood that any adequate method for analyzing images to recognize objects in images and therefore follow the position of objects from one image to another may be used. For example, any adequate machine learning models or deep learning models configured for recognizing objects/subjects within images may be used. For example, image segmentation and blob analysis may be used for identifying the needle and the subject within the sequence of images. In another embodiment, a convolution neural network (CNN) may be trained to recognize the needle and the subject within the sequence of images.

It should also be understood that any adequate method for determining that the needle comes into contact with the surface of the subject into an image may be used. For example, once the subject and the needle have been recognized and tracked in the images, the point of contact between the needle and the subject may be established when the distal end of the needle is positioned on the surface of the subject. For example, the position of the distal end of the needle may be tracked from one image to another and the point of contact between the needle and the subject is established when the coordinates of the distal end of the needle corresponds to the coordinates of one point of the surface of the subject.

In another example, a machine learning model such as a deep learning model may be trained to detect whether the distal end of a needle is in contact with the surface of a subject. In this case, the point of contact between the distal end of the needle and the surface of the subject is determined using the machine learning model.

In a further example, the point of contact between the distal end of the needle and the surface of the subject may be determined by calculating the distance between a reference point located on the needle or the syringe and the surface of the subject and comparing the calculated distance to a target or reference distance. When the calculated distance is equal to or less that the target distance, it is assumed that the distal end of the needle is in physical contact with the subject. In this case, the method 10 further comprises a step of receiving the target distance and optionally the identification of the reference point and.

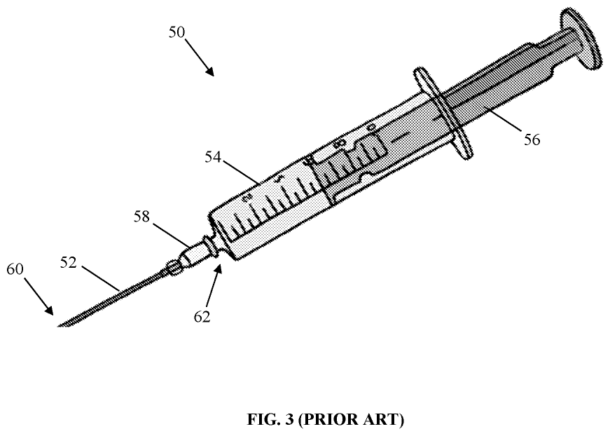

Referring to FIG. 3, there is illustrated an exemplary syringe 50 provided with a needle 52. As known in the art, the syringe 50 comprises an elongated and hollow barrel 54 and a plunger 56 insertable into the barrel 54. The needle 52 is fluidly connected to the barrel 54 via an adapter 58. For example, the reference point may be the distal end 60 of the needle 52. In another example such as when the diameter of the needle 52 is too small to be adequately visible in the images, the reference point may be the adapter 58 or the distal end of the adapter 58. In a further example, the reference point may be the distal end 62 of the barrel 54. It should be understood that in order to calculate the distance, at least one dimension of one of the elements present in the images must be known and the length of the needle must also be known.

In one embodiment, the distance between the reference point and the subject corresponds to the distance between the reference point and the surface of the subject along the longitudinal axis of the needle (which also corresponds to the longitudinal axis of the barrel 54). In another embodiment, the distance between the reference point and the subject corresponds to the shortest distance between the reference point and the surface of the subject.

Referring back to FIG. 1, once it has been detected that the needle came into contact with the subject, the insertion angle of the needle is calculated at step 16. The insertion angle corresponds to the angle between the needle and the subject as calculated from the images, i.e., the angle between the longitudinal axis of the needle/syringe and the tangent line to the surface of the subject at the contact point between the needle and the surface of the subject. It should be understood that any adequate method for calculating the insertion angle of the needle from the received images can be used.

In one embodiment, the insertion angle of the needle is calculated once only. For example, the first image of the sequence in which a point of contact between the needle and the subject is detected may be identified and the insertion angle may be calculated only in this first image.

In another embodiment, the insertion angle is iteratively calculated at several points in time (or in several images of the sequence) during the insertion of the needle within the subject. For example, the insertion angle may be calculated for each image following the detection of the point of contact between the needle and the subject. In one embodiment, the calculation of the insertion angle is stopped once a desired insertion depth is reached such as when the needle has been entirely inserted into the subject. In another embodiment, the calculation of the insertion angle is stopped once the syringe or the needle stops moving relative to the subject along the longitudinal axis of the syringe/needle.

At step 18, the depth of insertion of the needle into the subject is determined from the received images. It should be understood that any adequate method for determining the depth of insertion of the needle within the subject may be used. The insertion of the needle into the subject occurs from a first point in time (or a first image) at which the needle comes into contact with the surface of the subject until a second point in time (or a second image) at which the syringe stops moving relative to the subject along the longitudinal axis of the syringe. In one embodiment, the second point in time corresponds to the point in time at which the syringe has stopped moving relative to the subject along the longitudinal axis of the syringe for a predetermined period of time. The depth of insertion corresponds to the length of the portion of the needle that is inserted into the subject at the second point in time.

In one embodiment, the user is instructed to insert the whole needle into the subject. In this case, step 18 may consist in determining whether at the second point in time (or in the second image) the needle has been inserted entirely into the subject, i.e., whether the needle is visible or not in the second image corresponding to the second point in time. In this case, the insertion depth may have two values: “entirely inserted” and “partially inserted”. In another example, the insertion depth may have the two following values: “needle visible” and “needle not visible”. It should be understood that any adequate method for determining if a whole needle has been inserted into a subject from images or determining whether a needle is visible in images may be used.

In one embodiment, the needle is considered to be entirely inserted into the subject when a reference point comes into contact with the subject at the second point in time (i.e., the point in time at which the syringe stops moving relative to the subject along the longitudinal axis of the syringe or the point in time at which the syringe has stopped moving relative to the subject along the longitudinal axis of the syringe for a predetermined period of time). For example, turning to FIG. 3, the reference point may be located on the adapter 58 securing the needle 52 to the syringe 50. In another example, the reference point may correspond to the distal end 62 of the syringe 50. The image corresponding to the second point in time is analyzed to determine the position of the reference point in time relative to the surface. The insertion depth may then be calculated knowing the position of the reference point relative to the surface of the subject (which is equivalent to the distance between the reference point and the surface of the subject along the longitudinal axis of the syringe), the length of the needle and the position of the reference point relative to the needle.

In another embodiment, machine learning models such as deep learning models may be trained to determine whether a needle is entirely inserted into a subject. For example, the machine learning model may be trained to determine whether a reference point on the syringe or the adapter is in contact with the surface of the subject. The image taken at the second point in time is then analyzed by the machine learning model to determine whether the needle is entirely inserted into the subject. The machine learning model may analyze the received sequence of images and identify the first image in which the needle is entirely inserted into the subject. If no such image is identified by the machine learning model, then it is concluded that the needle was not entirely inserted into the subject. In another example, the machine learning model may analyze the received sequence of image, identify the first image at which the needle stops moving relative to the subject along the longitudinal axis of the syringe and determine whether the needle is visible in the first image. The machine learning model outputs the value “visible” if the needle is visible in the first image and the value “not visible” if the needle is not visible in the first image.

In a further embodiment, the depth of insertion of the needle is determined from the images by measuring the length of the visible portion of the needle within the images. A given image of the sequence in which the syringe has stopped moving relative to the subject along the longitudinal axis of the syringe is identified. The length of the visible portion of the needle within the identified image is determined and the insertion depth is calculated as being the length of the needle minus the determined length of the visible portion of the needle. If the needle is no longer visible, then it is assumed that the whole needle has been inserted into the subject and the insertion depth is equal to the length of the needle. In this embodiment, the method 10 further comprises the step of receiving the length of the needle.

In a further embodiment, the motion of a reference point located on the syringe or the adapter is tracked within the images starting from the first time at which the contact between the needle and the surface of the subject has been detected until the second point time at which the reference point stops moving relative the subject along the longitudinal axis of the syringe. As described above, the reference point may be located on the adapter securing the needle to the barrel of a syringe or on the syringe such at the distal end of the syringe. In one embodiment, the depth of insertion of the needle corresponds to the distance travelled by the reference point along the longitudinal axis of the syringe between the first and second points in time. In another embodiment in which the length of the needle is known, the distance between the reference point and the surface of the subject along the longitudinal axis of the syringe is determined at the second point in time and the depth of insertion of the needle within the subject can be determined from the determined distance and the length of the needle. For example, if the reference point is located on the adapter, then the needle is considered to be entirely inserted into the subject if the measured distance between the adapter and the surface of the subject is substantially equal to zero. In this embodiment, the method 10 further comprises a step of receiving an identification of the reference point.

Referring back to FIG. 1, the next step 20 consists in determining an injection speed or injection duration. The injection duration refers to the duration taken by the user to inject the given volume of pharmaceutical product within the subject. In one embodiment, the injection duration corresponds to the time difference between the point in time (or the image) at which the plunger starts moving and the point in time (or the image) at which the plunger stops moving. In another embodiment, the injection duration corresponds to the time elapsed during the motion of the plunger between two extreme positions relative to the syringe. The injection speed may refer to the speed at which the plunger is moving during the injection of the pharmaceutical product which is equivalent to the volume of pharmaceutical product delivered per unit of time. In one embodiment, the injection speed corresponds to the displacement speed of the plunger between the point in time at which the plunger starts moving and the point in time at which the plunger stops moving. In another embodiment, the injection speed corresponds to the displacement speed of the plunger during the motion of the plunger between two extreme positions relative to the syringe. The person skilled in the art will understand that the injection duration for a given volume of pharmaceutical product is equivalent to the injection speed since the greater the injection speed is, the shorter the time it takes to inject the pharmaceutical product.

In one embodiment, the speed of injection refers to the amount of pharmaceutical product injected per unit of time, such as per second. For example, knowing the diameter of the barrel of the syringe, the amount of pharmaceutical product injected per unit of time can be determined based on the displacement speed of the plunger or the injection duration. In this case, the method further comprises a step of receiving the diameter of the barrel of the syringe.

It should be understood that any adequate method for determining the injection duration or the injection speed may be used.

In one embodiment, the injection speed or the injection duration is determined from the received images. In another embodiment, a tracking system such as a triangulation tracking system may be used to determine and track the position of the plunger of the syringe. For example, the plunger may be provided with a signal emitting device configured for emitting a signal such as a radio frequency signal and sensors are used to detect the emitted signal. As known in the art, the position of the plunger, such as the position of the distal end of the plunger inserted into the barrel of the syringe, may then be determined from the signals received by the sensors. Knowing the position in time of the plunger, the injection duration, i.e., the time taken by the plunger to move between two extreme positions, and/or the speed of injection, i.e., the speed at which the plunger moves between the two extreme positions, may be determined.

In an embodiment in which the images are used to determine the injection duration, the injection duration may be assumed as being the period of time elapsed between the point in time at which the distal end of the needle came into contact with the subject and the subsequent point in time at which the needle is no longer in contact with the subject (i.e., the point in time at which the needle is extracted from the subject). In this case, the duration of the injection corresponds to the time elapsed between the first image at which the needle comes into contact with the subject and the first subsequent image at which the needle is no longer in contact with the subject. The injection speed may then correspond to the speed of displacement of the plunger during the motion of the plunger between the first image at which the needle comes into contact with the subject and the first subsequent image at which the needle is no longer in contact with the subject.

In another embodiment in which the images are used to determine the injection duration, the injection duration may be assumed as being the period of time elapsed between the first point in time at which the insertion depth has reached the desired depth and the first subsequent point in time at which the needle is no longer in contact with the subject. In this case, the duration of the injection corresponds to the time elapsed between the first image at which the insertion depth has reached the desired depth and the first subsequent image at which the needle is no longer in contact with the subject. The injection speed may then correspond to the speed of displacement of the plunger during the motion of the plunger between the first image at which the insertion depth has reached the desired depth and the first subsequent image at which the needle is no longer in contact with the subject.

In a further embodiment in which the images are used to determine the injection duration or the injection speed, the injection duration or the injection speed is determined based on the position in time of the plunger relative to the barrel. For example, a reference point on the plunger, such as the distal end of the plunger, may be localized within the images and the motion of the distal end of the plunger relative to the barrel may be tracked between its two extreme positions while the pharmaceutical product is injected. By tracking the position of the distal end of the plunger, the injection duration and/or the injection speed may be determined.

In one embodiment, at least one portion of the plunger such as the distal end of the plunger or plunger head of the plunger may be provided with a predefined color so as to allow an easier localization of the plunger within the images. The extremities of the barrel may also be provided with a respective color while still being translucent and the portion of the barrel extending between the two extremities may be substantially transparent. In this case, when the plunger head moves away from the proximal extremity, the color of the proximal extremity is revealed. Conversely, as the plunger head reaches the distal extremity, the color of the distal extremity as perceived by the camera changes. The position of the plunger head relative to the barrel may then be approximated by merely detecting the changes in color of the barrel extremities.

At step 22, the insertion angle, the insertion depth and the injection duration or speed are outputted. For example, they may be stored in memory. In another example, they may be provided for display on a display unit. In a further example, they may be transmitted to a computer machine.

In one embodiment, the method 10 further comprises a step of evaluating the injection parameters, i.e., the determined insertion angle, insertion depth and injection duration or speed.

In one embodiment, the determined insertion angle is compared to two angle thresholds, i.e., a minimal angle and a maximal angle. If the insertion angle is comprised between the minimal and maximal angles, then the insertion angle is identified as being adequate. Otherwise, the insertion angle is identified as being inadequate.

In an embodiment in which the insertion angle is determined at different points in time during the insertion of the needle, each determined insertion angle may be compared to the minimal and maximal insertion angles. If at least one of the determined insertion angles is not comprised between the minimal and maximal insertion angles, then the insertion of the needle may be considered as being inadequate. If all of the determined insertion angles are comprised between the minimal and maximal insertion angles, then the insertion of the needle is considered to be adequate. In another example, the median or the mean of the different determined insertion angles may be compared to the minimal and maximal insertion angles. If the median or the mean of the different determined insertion angles is not comprised between the minimal and maximal insertion angles, then the insertion of the needle may be considered as being inadequate. Otherwise, the insertion of the needle is considered to be adequate.

In one embodiment, the determined insertion depth is compared to at least one depth threshold and the determined insertion depth is identified as being adequate or inadequate based on the comparison. For example, the determined insertion depth may be compared to a minimal depth. If the determined insertion depth is less than the minimal depth, then the determined depth is considered as being inadequate. Otherwise, the determined depth is considered as being adequate.

In an embodiment in which the needle has to be entirely inserted into the subject for the insertion to be adequate and the step 18 of determining the insertion depth consists in determining whether the needle is entirely inserted into a subject, the output value of step 18 is compared to a target value, e.g., “entirely inserted” or “not visible”. If the output value of step 18 corresponds to the target value, then the insertion depth is considered as being adequate. Otherwise, if the output value of step 18 does not correspond to the target value, then the insertion depth is considered as being inadequate. For example, if the two possible output values for step 18 are “visible” and “not visible”, the target value is “visible” and the actual output value determined at step 18 is “not visible”, then it is determined that the insertion depth is inadequate. However, if the actual output value determined at step 18 is “visible”, then it is determined that the insertion depth is adequate.

In one embodiment, the determined injection duration or speed is compared to at least one injection threshold. For example, in an embodiment in which the injection duration is determined at step 20, the determined injection duration may be compared to a minimal duration. If the determined injection duration is less than the minimal duration, the injection duration is identified as being inadequate. Otherwise, the injection duration is identified as being adequate. For example, in an embodiment in which the injection speed is determined at step 20, the determined injection speed may be compared to a maximal duration. If the determined injection speed is greater than the maximal speed, the injection speed is identified as being inadequate. Otherwise, the injection speed is identified as being adequate.

Once the parameters of the injection have been evaluated, the evaluation results are outputted, i.e., once the determined insertion angle, the determined insertion depth and the determined injection duration or speed have been evaluated, an indication as to whether the determined insertion angle, the determined insertion depth and the determined injection duration or speed are adequate or not is outputted. For example, the evaluation results may be stored in memory. In another example, they may be provided for display on a display unit.

In one embodiment, the method 10 further comprises the step of capturing the sequence of images using a camera.

In one embodiment, the steps 14 to 20 are performed in substantially real-time while the images are being acquired.

In one embodiment, the evaluation of the determined insertion angle, insertion depth and injection duration or speed is performed in substantially real-time while the camera acquires the images. In this case, the injection parameters are evaluated as the images are received. In this case, a substantially real-time feedback can be provided to the user. For example, when it is detected that the needle came into contact with the subject, the insertion angle may be determined and evaluated and an indication as to whether the insertion angle is adequate can be provided to the user, thereby allowing the user to correct the insertion angle in the event the determined insertion angle is considered to be inadequate. In another embodiment, the

[symbol]

In one embodiment, the method 10 may be used for training a user such as a medical student without the presence of a trainer such as a professor, a supervisor or the like. In this case, the user records a video while performing an injection and the video is transmitted to a computer machine such as a server that executes the method 10 to calculate the injection parameters and optionally evaluate the injection parameters. Hence, a user may be evaluated without requiring the presence of a trainer.

As described above, the method 10 may be used when an inanimate subject is used. In this case, the pharmaceutical product may be air for example. Such a scenario is particularly adequate for training users, especially training users remotely.

In an embodiment in which the subject is a living subject, the method 10 may be used for evaluating medical staff members such as nurses and allowing them to improve their skills.

In one embodiment, the method 10 may be embodied as a non-transitory memory having stored thereon statements and instructions that when executed by a processing unit perform the steps of the method 10.

In another embodiment, the method 10 may be embodied as a system comprising at least one processing unit configured for performing the steps of the method 10.

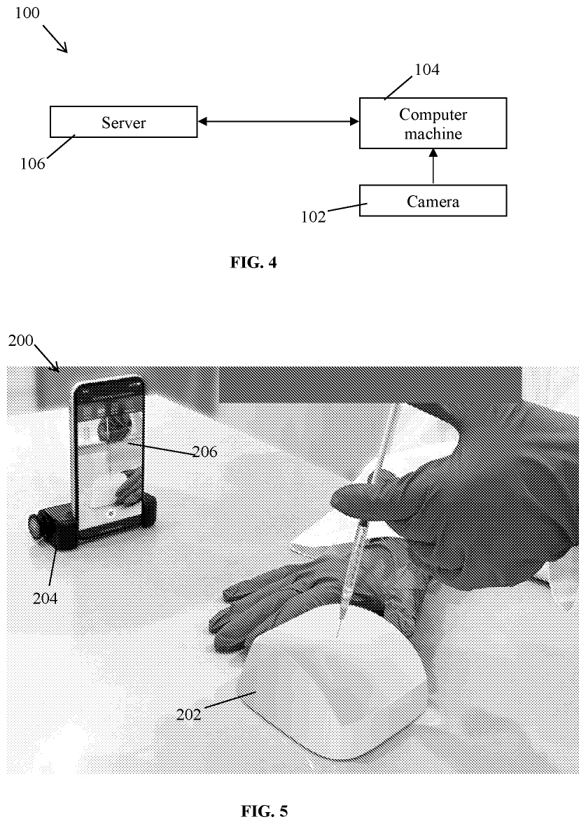

FIG. 4 illustrates one embodiment of a system 100 for assessing an injection of a pharmaceutical product on a subject while using a syringe provided with a needle. The system comprises a camera 102 for capturing images of the subject and the syringe while a user performs the injection of the pharmaceutical product, a computer machine 104 connected to the camera 102 and a server 106 for calculating and evaluating the injection parameters.

The camera 102 captures a video of the injection which is transmitted to the computer machine 104. In one embodiment, the camera 102 may be integral with the computer machine 104 such as when the computer machine 104 is a laptop, a smartphone, a tablet, or the like.

In one embodiment, the computer machine 104 receives the video and transmits the received video to the server 106 over a communication network such as the Internet. In this case, the server 106 is configured for performing the steps of the method 10. For example, the server 106 may be configured to perform the evaluation of the injection. The evaluation results may be stored in memory by the server 106 and/or displayed on a display connected to the server 106. The server 106 may also transmits the evaluation results to the computer machine 104 which may provide the received evaluation results for display on a display connected to the computer machine 104.

In one embodiment, the server 106 may be omitted and the computer machine 104 is configured for performing the steps of the method 10.

In the following there is described a system 200 for assessing an injection of a pharmaceutical product on a subject while using a syringe provided with a needle and using a smartphone or a tablet.

As illustrated in FIG. 5, the system 200 comprises an inanimate subject, i.e., the anatomical model 202 on which the insertion of the needle is to be performed and a support 204 for receiving a smartphone 206 therein.

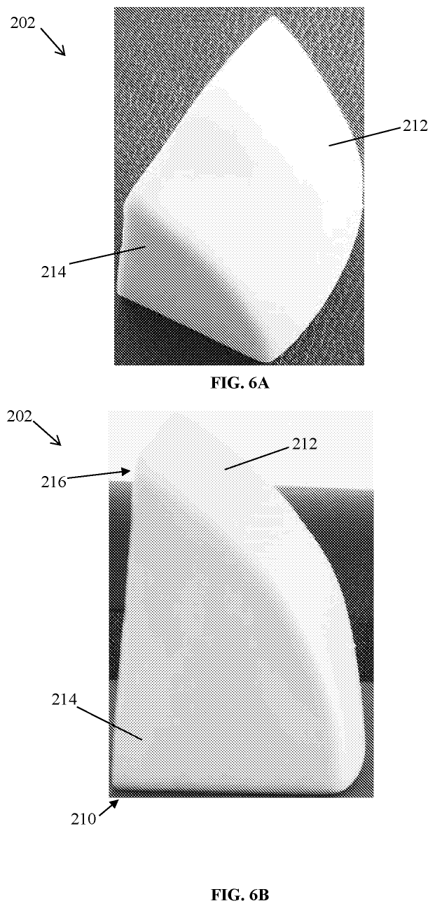

In the illustrated embodiment, the anatomical model 202 mimics the shape of a portion of a shoulder. As illustrated in FIGS. 6A and 6B, the anatomical model 202 comprises a body having a bottom face 210 configured for abutting a receiving surface on which the anatomical model is to be deposited, a working face 212, two lateral faces 214 and a back face 216. The two lateral faces 216 are planar and parallel to each other. In the illustrated embodiment, the lateral faces 214 are orthogonal to the bottom face 210. The working face 212 extends laterally between the two lateral faces 214 and longitudinally between the bottom face 210 and the back face 216. The working face 212 is the face in which the needle is to be inserted and is provided with an elliptical shape so as to mimic the shape of a shoulder.

It should be understood that the anatomical model 202 is made of any adequate material allowing the insertion of a needle therein. For example, the anatomical model 202 may be made of foam.

Referring back to FIG. 5, the support 204 is designed and shaped to include a recess or opening in which the smartphone 206 may be received and held in position. In the illustrated embodiment, the support 204 is designed so that the smartphone 206 be substantially orthogonal to the surface on which the support 204 is positioned.

The support 204 is positioned relative to the anatomical model 202 so that the anatomical model 202 be in the field on view of the camera of the smartphone 206. In one embodiment, the relative positioning of the support 204 and the anatomical model 202 is chosen so that one of the lateral faces 214 is parallel to the plane of the camera of the smartphone when the smartphone is received in the support 204, i.e., parallel to the plane in which the front face of the smartphone extends.

In one embodiment, the support 204 is shaped and sized to provide a predefined orientation of the plane of the camera of the smartphone 206 relative to the surface on which the support 204 is positioned. For example, the support 204 may be shaped and sized so that the plane of the camera, e.g., the plane in which the smartphone extends, be orthogonal to the surface on which the support 204 is deposited.

In one embodiment, the support 204 is positioned at a predefined distance from the anatomical model 202. In one embodiment, the system 200 further comprises a mat on which the anatomical model 202 and the support 204 are to be installed. The mat is provided with reference marks thereon to help the user adequately position and orient the anatomical model 202 and the support 204 relatively to one another. For example, the mat may comprise a first mark thereon for adequately positioning the anatomical model 202 and a second mark thereon for adequately positioning the support 204 Proper use of the mat ensures that the anatomical model 202 and the support 204 are at a predefined distance from one another and the smartphone 206 when received in the support 204 is adequately oriented relative to a lateral face 214 of the anatomical model 202.

The smartphone 206 is provided with an application stored thereon as described below. The application is configured for guiding the user to install the smartphone 206 into the support 204 and position and orient the support 204 relative to the anatomical model 202. The application is further configured for receiving the video of the injection captured by the camera of the smartphone 206 and transmit the received video of the injection to a server such as the server 106 which executes the steps of the method 10. The evaluation results of the injection parameters generated by the server are transmitted to the smartphone 206 which displays the received results on its display.

In one embodiment, the application is configured for transmitting the images captured by the camera as they are captured by the camera. In another embodiment, the application is configured for transmitting the images only when the recording of the video ended.

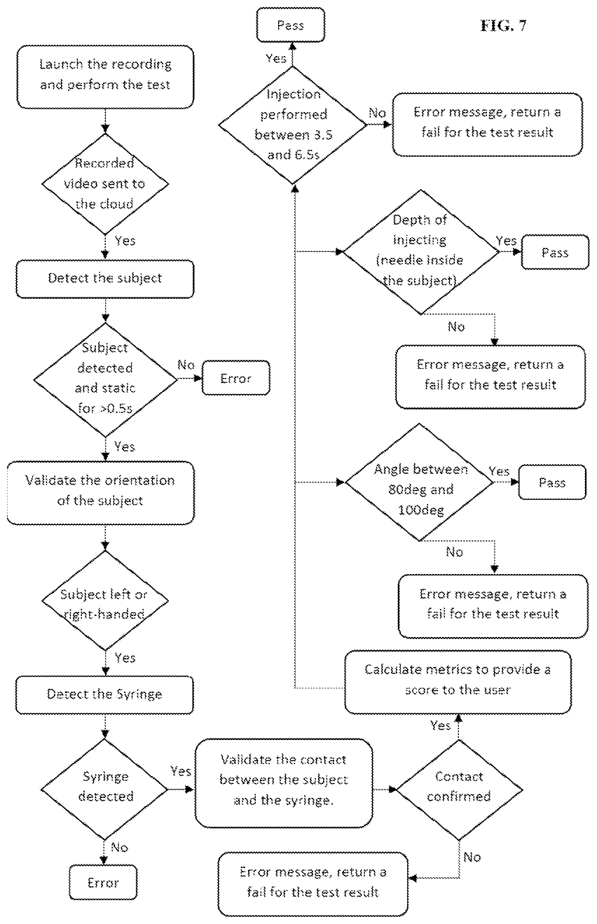

FIG. 7 is a flow chart illustrating exemplary steps performed by the smartphone 206 and the server for assessing a recorded injection.

Prior to the execution of the flow chart of FIG. 7, the application running on the smartphone 202 is configured for collecting information from the user.

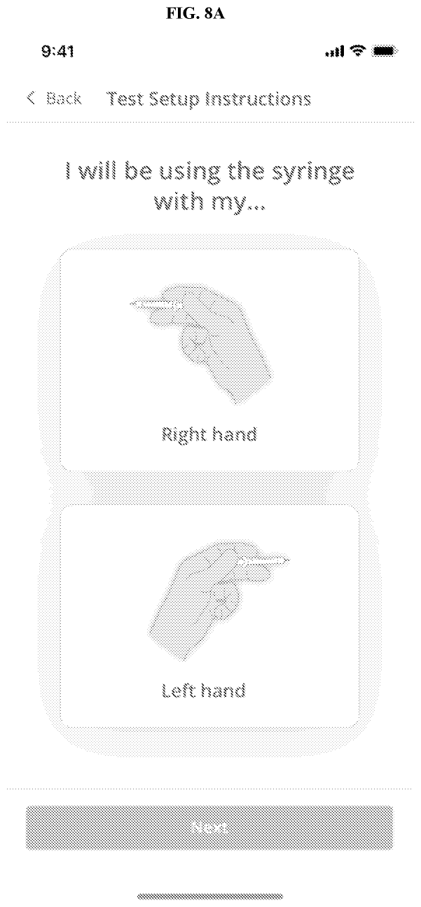

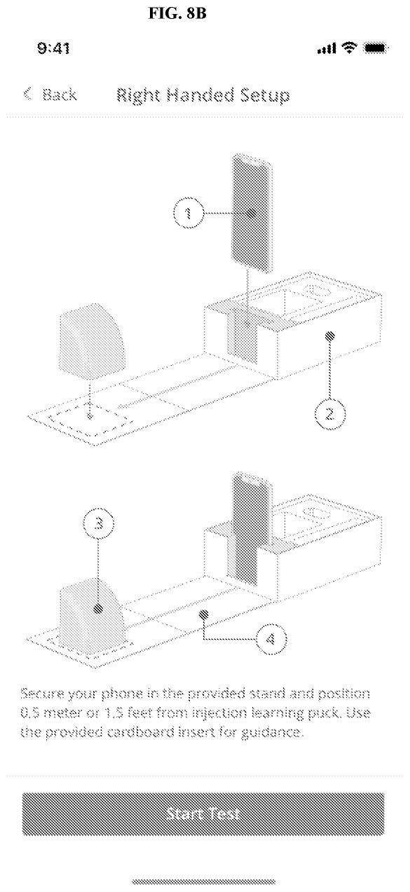

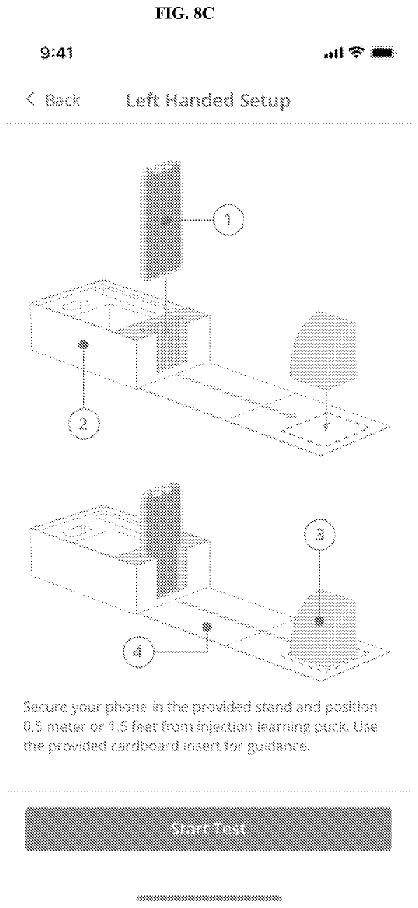

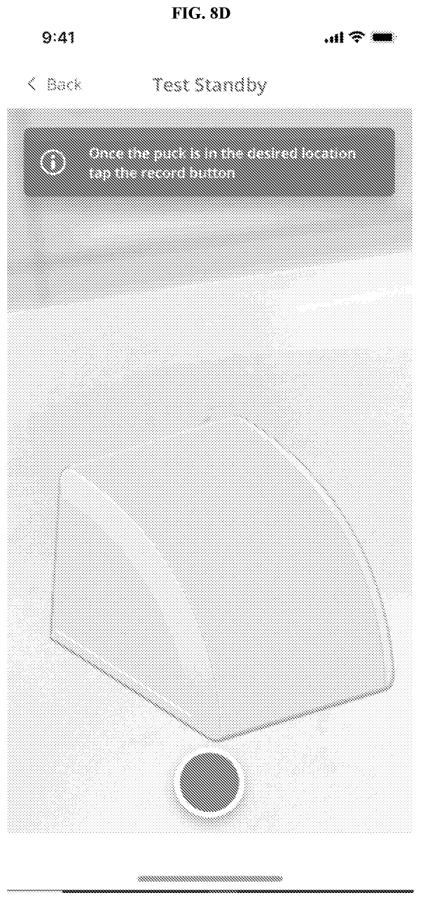

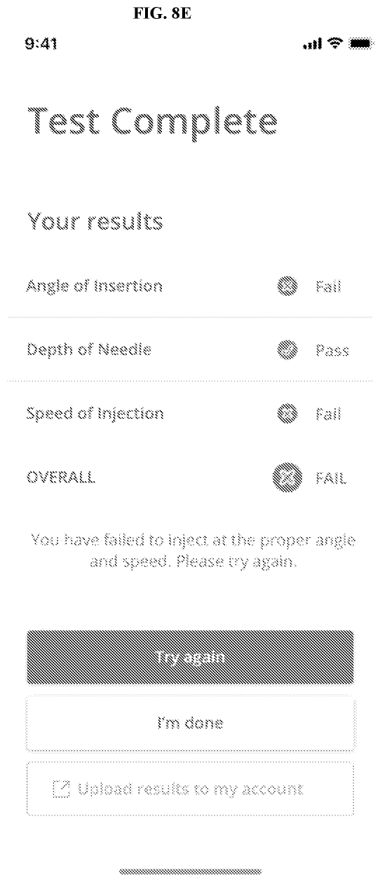

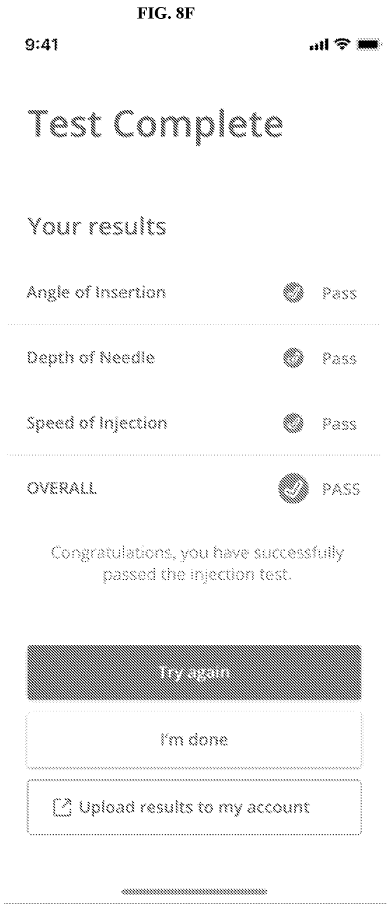

FIGS. 8A-8C illustrates exemplary interfaces that may be generated by the application and displayed on the screen of the smartphone 206. FIG. 8A illustrates an exemplary interface adapted to ask the user whether the injection will be performed with the right hand or the left hand. If the user indicates within the interface that the right hand will be used, the application displays the exemplary interface of FIG. 8B while the interface of FIG. 8C is displayed if the user indicates the left hand will be used.

The interface of FIG. 8B illustrates the adequate setup for a right-handed user and guides the user to adequately install the anatomical model 202 and the support 204 on the mat, install the smartphone 206 into the support 204 and adequately orient the working face 212 relative to the smartphone 206

The interface of FIG. 8C illustrates the adequate setup for a left-handed user and guides the user to adequately install the anatomical model 202 and the support 204 on the mat, install the smartphone 206 into the support 204 and adequately orient the working face 212 relative to the smartphone 206.

Once the user indicates that the installation is completed, the application displays the exemplary interface of FIG. 8D which informs the user that he may start recording and perform the injection on the anatomical model 202. The method of FIG. 7 is then executed.

As illustrated in FIG. 7, the smartphone records the video of the user performing the injection and transmits the recorded video to the server which may be located in the cloud for example. The server analyses the received frames of the video to detect the subject. It should be understood that the frames of the video form a sequence of images and the server iteratively analyses the frames according to their order in the sequence.

The first step of the analysis performed by the server is the detection of the subject or anatomical model 202 within the video frame. Once the anatomical model 202 has been detected, a verification step is performed, i.e., the server ensure that the detected object identified as the anatomical model 202 does not move for a given period of time such as at least 0.5 second. If the object identified as the anatomical model 202 moves during the period of time, the server understands that the identified object is not the anatomical model 202 and transmits an error message to the smartphone to be displayed thereon. For example, the error message may indicate that the anatomical model 202 has to be placed at the location indicated on the mat.

Once the anatomical model 202 has been identified, the orientation of the working surface 212 of the anatomical model 202 is detected and validated with the information inputted by the user regarding whether he is right or left-handed.

Then the syringe is detected within the frames of the video. If the syringe is not detected in the video frames, an error message is generated by the server and transmitted to the smartphone 206 to be displayed thereon. For example, the error message may inform the user that the syringe was not detected and request the user to ensure that proper lightning of the room is used.

Once the syringe has been detected, the server identifies the first frame in which the distal end of the needle comes into contact with the surface of the anatomical model 202. If it cannot detect a contact between the needle and the anatomical model 202, the server generates an error message and transmits the error message to the smartphone 206 to be displayed thereon. The error message may indicate that no contact between the needle and the anatomical model 202 was detected and request the user to ensure that proper lighting of the room is used.

Once the contact between the needle and the anatomical model 202 has been detected, the server analyses the subsequent frames to calculate the injection parameters, i.e. the insertion angle of the needle, the depth of insertion and the injection duration/speed. The server then compares the calculated parameters to respective thresholds, as described above.

If the calculated insertion angle is not adequate, the server generates and transmits to the smartphone 206 an error message to be displayed thereon. For example, when the insertion angle is not comprised between a given range such as between 80 degrees and 100 degrees, the error message may indicate that the insertion angle falls outside the recommended range.

If the calculated insertion depth is not adequate, the server generates and transmits to the smartphone 206 an error message to be displayed thereon. For example, when the needle has to be entirely inserted into the anatomical model 202 and the server determines that the needle was not entirely inserted, the error message may indicate that the needle needs to be fully inserted into the anatomical model 202.

If the calculated injection duration is not adequate, the server generates and transmits to the smartphone 206 an error message to be displayed thereon. For example, when the injection duration is not comprised between a given range such as between 3.5 seconds and 6.5 seconds, the error message may indicate that the insertion speed was too fast or too slow.

If the calculated insertion angle, insertion depth and injection duration/speed are each found to be adequate, the server generates and transmits to the smartphone 206 an evaluation message indicating that all injection parameters were found adequate.

The application running on the smartphone may generate a graphical interface for displaying the results of the evaluation. FIG. 8E illustrates an exemplary interface that may be used for informing the user that he successfully inserted the entire needle into the anatomical model 202 but failed to insert the needle at an adequate insertion angle and perform the injection at an adequate speed. FIG. 8F illustrates an exemplary interface that may be used for informing the user that he successfully performed the injection.

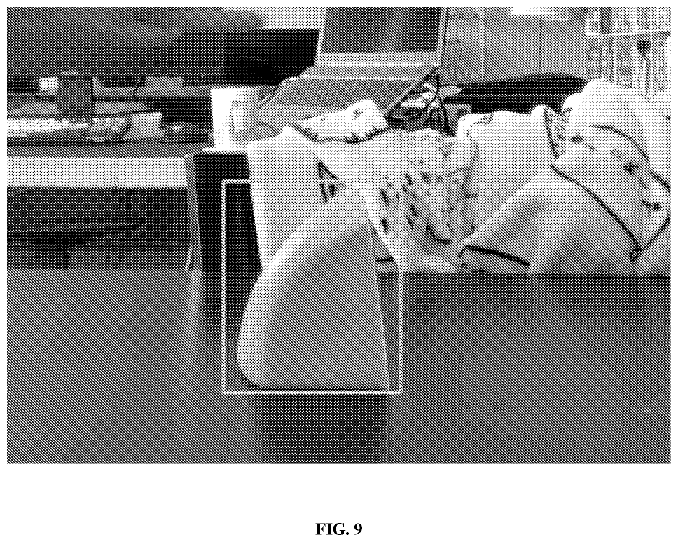

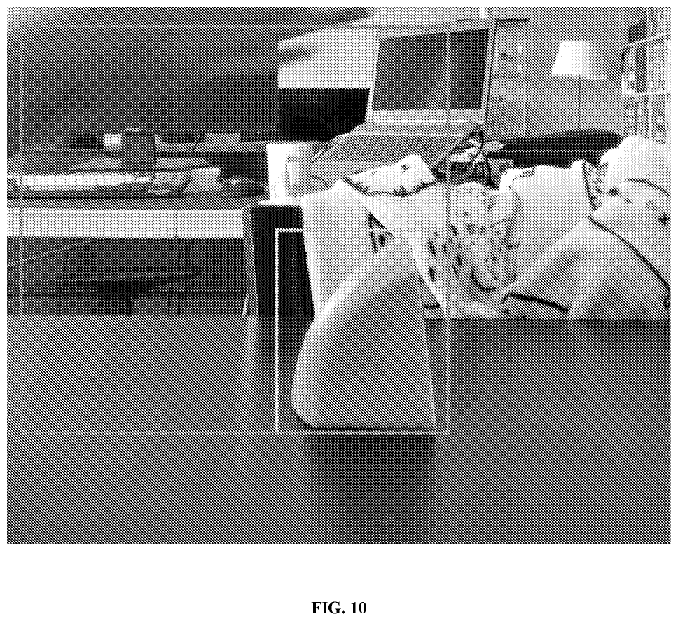

It should be understood that the server may execute any adequate methods or machine learning models configured for object recognition and tracking to locate and identify the anatomical model 202 and the syringe. For example, GOTURN or Kernelized Correlation Filters may be used. In another example, a machine learning model such as Yolo RetinaNet, SSD, Fast RCNN, or the like may be used. In one embodiment, a bounding box is generated around the anatomical model 202 as illustrated in FIG. 9.

It should also be understood that any adequate method may be used for determining the orientation of the working face 212 of the anatomical model 202. In one embodiment, the user may be requested to place at least one hand within the field of view of the camera and to the side of the working face 212. The orientation of the working face 212 may then be determined using the location of the hand(s) within the images. The detection of the hand(s) by the server may be performed using methods such as Histogram Oriented Gradient, Canny edge detector and/or Support Vector Machine, or a trained machine learning model such as DeepPose.

In another example, the determination of the orientation of the working face 212 may be automatically performed by the server. For example, the server may execute methods such as Histogram Oriented Gradient and Support Vector Machine to determine the orientation of the working face 212. In another example, a machine learning model such as a CNN with a binary classification (i.e. right or left) may be trained to determine the orientation of the working face 212.

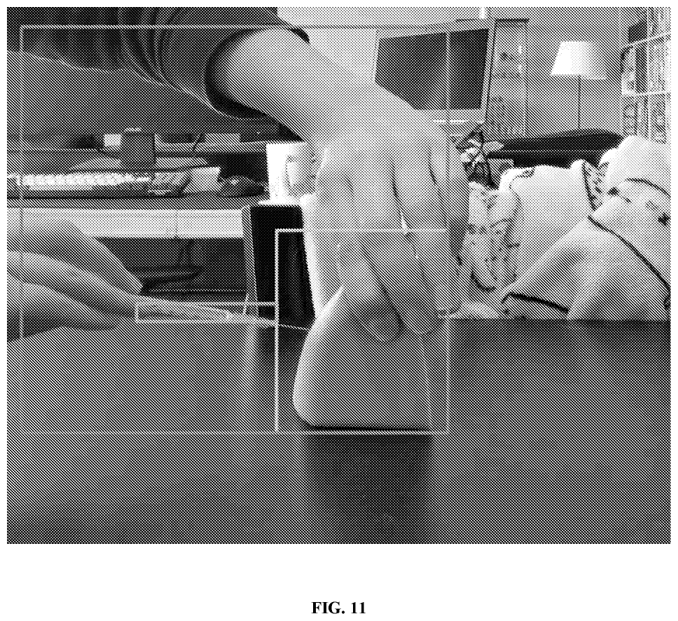

In one embodiment, once the working face 212 of the anatomical model 202 has been detected, a second bounding box is generated around the anatomical model 202 as illustrated in FIG. 10. The second bounding box is larger than the bounding box identifying the anatomical model 2020 and extends on the side of the working face 212 of the anatomical model 202. The second bounding box represents a search area in which the syringe should be located.

Once the syringe has been detected, a bounding box is assigned to the syringe, as illustrated in FIG. 11. In the illustrated FIGURE, the distal end of the needle comes into contact with the anatomical model 202. The contact between the needle and the anatomical model 202 is detected using a machine learning model previously trained using labeled pictures showing a contact and labeled pictures showing non-contact to determine whether a contact between a needle and an anatomical model exists.

Once the contact has been determined, the server determines the insertion angle by determining the tangent to the surface of the anatomical model 202 at the contact point and calculating the angle between a first vector oriented along the longitudinal axis of the syringe and a second vector oriented along the determined tangent. However, it should be understood that any adequate method for calculating the insertion angle may be used. For example, the insertion angle may be assumed as corresponding to the angle of the diagonal of the bounding box associated with the syringe. In another example, Histogram Oriented Gradient may be used for calculating the insertion angle. In a further example, Principal Component Analysis (PCA) may be used.

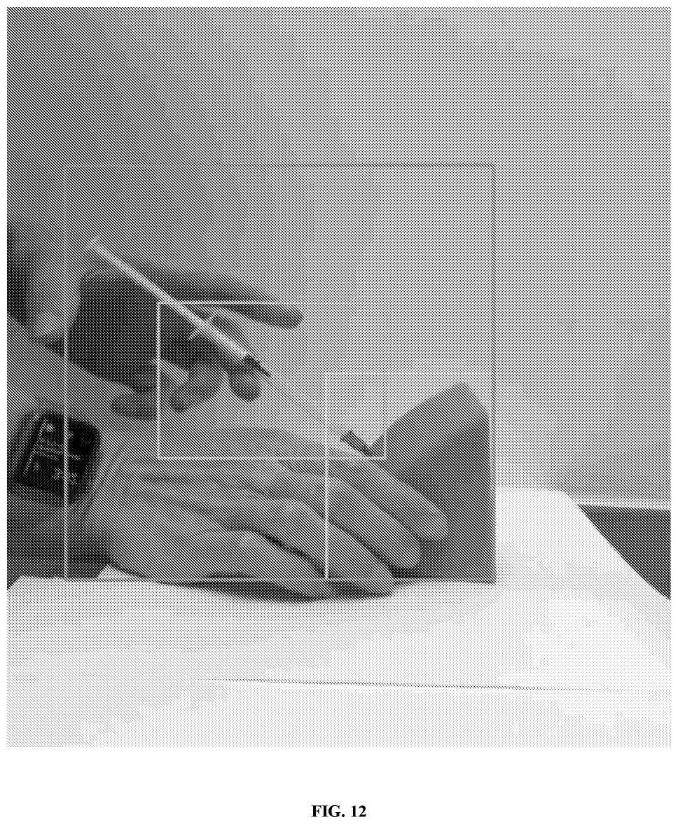

Then the server determines when the needle has been entirely inserted into the anatomical model 202, as illustrated in FIG. 12. The server executes a machine learning model trained to determine whether the needle has been completely inserted into the anatomical model 202. The machine learning model is previously trained using labeled pictures showing a completely inserted needle and labeled pictures showing a partially inserted needle to determine whether a needle is entirely inserted.

While the above methods and systems are directed to the assessment of the injection of a pharmaceutical product while using a needle and a syringe, it should be understood that the methods and system may be adapted for the assessment of a blood withdrawal and a joint aspiration for example. In this case, the step of determining the depth of insertion and/or the step of determining the speed or duration of injection may be omitted.

The embodiments of the invention described above are intended to be exemplary only. The scope of the invention is therefore intended to be limited solely by the scope of the appended claims.

I/We claim:

1. A computer-implemented method for assessing an injection performed on a subject using a syringe provided with a needle, the method comprising:
processing a sequence of images taken of the injection for:
determining an insertion angle of the needle relative to the subject; and
determining a depth of insertion of the needle within the subject;
determining one of a speed of injection and a duration of injection; and
outputting an indication of the insertion angle, the depth of insertion and the one of the speed of injection and the duration of injection.

2. The computer-implemented method of claim 1, wherein the insertion angle comprises an angle between a longitudinal axis of the syringe and a tangent line to a surface of the subject at a contact point between the needle and the surface of the subject.

3. The computer-implemented method of claim 2, wherein said determining an insertion angle comprises:
identifying a given one of the images in which a distal end of the needle comes into contact with the subject; and
measuring the insertion angle within the given one of the images.

4. The computer-implemented method of claim 3, wherein said identifying the given one of the images comprises processing the sequence of images for at least one of:
tracking the needle and the subject within the sequence of images, and
identifying the given one of the images as being a first image in the sequence of images in which first coordinates of the distal end of the needle corresponds to second coordinates of a point of the surface of the subject; and,
calculating a distance between a reference point located on one of the needle and the syringe and the surface of the subject, and
identifying the given one of the images as being a first image in the sequence of images in which the calculated distance is one of equal to and less than a target distance.

5. (canceled)

6. The computer-implemented method of claim 1, wherein said determining an insertion angle comprises:
identifying a plurality of the images in which a distal end of the needle comes into contact with the subject;
measuring the respective insertion angle within each one of the plurality of the images; and
calculating one of a median insertion angle and an average insertion angle based on the respective insertion angles, thereby obtaining the insertion angle.

7. (canceled)

8. The computer-implemented method of claim 1, wherein said determining a depth of insertion comprises processing the sequence of images for determining a given image in the sequence in which the needle has stopped moving along a longitudinal axis of the syringe.

9. The computer-implemented method of claim 8, wherein said determining a depth of insertion further comprises at least one of:
determining within the given image whether the needle is visible; and
measuring within the given image, a length of a visible portion of the needle; and
determining within the given image, a position of a reference point on the syringe relative to a surface of the subject and calculating the depth of insertion based on the position of the reference point.

10. (canceled)

11. (canceled)

12. The computer-implemented method of claim 1, wherein when said determining one of the speed of injection and the duration of injection comprises determining the duration of the injection, the duration of the injection corresponds to one of:
a time elapsed between a first image in which the needle comes into contact with the subject and a subsequent image in which the needle is no longer in contact with the subject; and
a time elapsed between a first image in which the insertion depth has reached a desired depth and a subsequent image in which the needle is no longer in contact with the subject.

13. (canceled)

14. (canceled)

15. The computer-implemented method of claim 1, wherein said determining one of the speed of injection and the duration of injection comprises tracking a position of a plunger relative to a barrel of the syringe.

16. (canceled)

17. The computer-implemented method of claim 1, further comprising:
determining whether the insertion angle is adequate, whether the depth of insertion is adequate and whether the one of the speed of injection and the duration of injection is adequate, thereby obtaining assessment results; and
outputting the assessment results;
wherein at least one of:
the insertion angle is determined as being adequate if the insertion angle is comprised between a predefined minimal angle and a predefined maximal angle;
the depth of insertion is determined as being adequate by determining that the syringe comes into contact with the subject;
the depth of insertion is determined as being adequate if the depth of insertion is comprised between a predefined minimal insertion and a predefined maximal insertion; and
the one of the speed of injection and the duration of injection is determined as being adequate if the one of the speed of injection and the duration of injection is comprised between a first threshold and a second threshold.

18. (canceled)

19. (canceled)

20. (canceled)

21. (canceled)

22. A system for assessing an injection of a pharmaceutical product, the system comprising at least one processor and a memory, the memory having stored thereon statements and instructions that upon execution by the at least one processor perform the steps of:
processing a sequence of images taken of the injection for:
determining an insertion angle of the needle relative to the subject; and
determining a depth of insertion of the needle within the subject;
determining one of a speed of injection and a duration of injection; and
outputting an indication of the insertion angle, the depth of insertion and the one of the speed of injection and the duration of injection.

23. The system of claim 22, wherein the insertion angle comprises an angle between a longitudinal axis of the syringe and a tangent line to a surface of the subject at a contact point between the needle and the surface of the subject.

24. The system of claim 23, wherein said determining an insertion angle comprises:
identifying a given one of the images in which a distal end of the needle comes into contact with the subject; and
measuring the insertion angle within the given one of the images.

25. The system of claim 24, wherein said identifying the given one of the images comprises processing the sequence of images for at least one of:
tracking the needle and the subject within the sequence of images, and
identifying the given one of the images as being a first image in the sequence of images in which first coordinates of the distal end of the needle corresponds to second coordinates of a point of the surface of the subject; and,
calculating a distance between a reference point located on one of the needle and the syringe and the surface of the subject, and
identifying the given one of the images as being a first image in the sequence of images in which the calculated distance is one of equal to and less than a target distance.

26. (canceled)

27. The system of claim 22, wherein said determining an insertion angle comprises:
identifying a plurality of the images in which a distal end of the needle comes into contact with the subject;
measuring the respective insertion angle within each one of the plurality of the images; and
calculating one of a median insertion angle and an average insertion angle based on the respective insertion angles, thereby obtaining the insertion angle.

28. (canceled)

29. The system of claim 22, wherein said determining a depth of insertion comprises processing the sequence of images for determining a given image in the sequence in which the needle has stopped moving along a longitudinal axis of the syringe.

30. The system of claim 29, wherein said determining a depth of insertion further comprises at least one of:
determining within the given image whether the needle is visible;
measuring within the given image, an length of a visible portion of the needle; and
determining within the given image, a position of a reference point on the syringe relative to a surface of the subject and calculating the depth of insertion based on the position of the reference point.

31. (canceled)

32. (canceled)

33. The system of claim 22, wherein when said determining one of the speed of injection and the duration of injection comprises determining the duration of the injection, the duration of the injection corresponds to one of:
a time elapsed between a first image in which the needle comes into contact with the subject and a subsequent image in which the needle is no longer in contact with the subject; and
a time elapsed between a first image in which the insertion depth has reached a desired depth and a subsequent image in which the needle is no longer in contact with the subject.

34. (canceled)

35. (canceled)

36. The system of claim 22, wherein said determining one of the speed of injection and the duration of injection comprises tracking a position of a plunger relative to a barrel of the syringe.

37. (canceled)

38. The system of claim 22, wherein the at least one processor is further configured for:
determining whether the insertion angle is adequate, whether the depth of insertion is adequate and whether the one of the speed of injection and the duration of injection is adequate, thereby obtaining assessment results; and
outputting the assessment results;
wherein at least one of:
the depth of insertion is determined as being adequate by determining that the syringe comes into contact with the subject;
the insertion angle is determined as being adequate if the insertion angle is comprised between a predefined minimal angle and a predefined maximal angle;
the depth of insertion is determined as being adequate if the depth of insertion is comprised between a predefined minimal insertion and a predefined maximal insertion; and
the one of the speed of injection and the duration of injection is determined as being adequate if the one of the speed of injection and the duration of injection is comprised between a first threshold and a second threshold.

39. (canceled)

40. (canceled)

41. (canceled)

42. (canceled)

43. (canceled)

44. A kit for assessing an injection performed using a syringe provided with a needle, the kit comprising:
a subject comprising an anatomical model;
a support comprising an opening for receiving therein a camera configured for capturing a sequence of images of the injection performed on the anatomical model; and
a syringe provided with a needle.)

45. (canceled)

46. (canceled)

47. (canceled)

48. (canceled)
